(19) United States
(12) Patent Application Publication
(10) Pub. No.: US 2024/0189425 A1
(43) Pub. Date: Jun. 13, 2024

(54) CHIMERIC ANTIGEN RECEPTOR WITH MODIFIED HINGE AND TRANSMEMBRANE DOMAIN AND USES THEREOF

(71) Applicant: Orgenesis Inc., Germantown, MD (US)

(72) Inventors: BUO CHEN, KATY, TX (US); Xiangqun LI, Beijing (CN)

(73) Assignee: Orgenesis Inc., Germantown, MD (US)

(21) Appl. No.: 18/583,721

(22) Filed: Feb. 21, 2024

Related U.S. Application Data
(63) Continuation of application No. PCT/US23/06915, filed on Jun. 23, 2023.
(60) Provisional application No. 63/355,538, filed on Jun. 24, 2022.
(60) Provisional application No. 63/382,926, filed on Nov. 9, 2022.

(51) Int. Cl. A61K 39/00; C07K 14/725; C07K 14/735; C07K 16/28; C12N 5/0783

(52) U.S. Cl. CPC A61K 39/464412; A61K 39/4611; A61K 39/4631; C07K 14/7051; C07K 14/70535; C07K 16/2803; C12N 5/0636; C07K 2317/622; C07K 2319/02; C07K 2319/03; C07K 2319/30; C12N 2510/00

(57) ABSTRACT

The present disclosure provides a chimeric antigen receptor (CAR) molecule comprising a modified hinge and transmembrane domain (HTM-1), wherein the modified hinge and transmembrane domain comprises the amino acid sequence of SEQ ID NO:2. Cells expressing CAR with the modified HTM-1 domain disclosed herein are expected to have enhanced anti-tumor activities with reduced release of pro-inflammatory cytokines.

### CROSS-REFERENCE TO RELATED APPLICATIONS

This application is a Continuation Application of PCT International Application No. PCT/US23/69015, International Filing Date Jun. 23, 2023 claiming priority of U.S. Provisional Patent Applications, 63/355,538 filed Jun. 24, 2022 and 63/382,926 file Nov. 9, 2022 all of which are incorporated herein by reference in their entirety.

### SEQUENCE LISTING

The instant application contains a Sequence Listing which has been submitted electronically in XML file format and is hereby incorporated by reference in its entirety. Said XML copy, created on Jun. 23, 2023, is named P-610175-PC_SL.xml and is 15,353 bytes in size.

### FIELD OF THE INVENTION

The present disclosure is related in general to constructs of chimeric antigen receptor (CAR). In one embodiment, the present disclosure provides improved CAR with modified hinge and transmembrane regions.

### BACKGROUND OF THE INVENTION

Chimeric antigen receptors (CARs) are recombinant receptors that provide both antigen-binding and T cell activating functions. CAR T cell therapy, named “Advance of the Year” in 2018 by the American Society of Clinical Oncology, has revolutionized cancer treatment. KYMRIAH® (tisagenlecleucel, Novartis) and YESCARTA® (axicabtagene ciloleucel, Gilead) were rapidly approved by the U.S. Food and Drug Administration, and the number of active clinical trials testing CAR T cells in patients has exploded. Currently, there is substantial interest in improving the efficacy of CAR T cell therapy, e.g. (i) minimizing the toxic side effects of hematologic malignancy-targeted CAR T cells and (ii) improving the efficacy of solid tumor-targeted CAR T cells.

CARs consist of molecules in which tumor antigen recognition and intracellular activation are combined. In general, they minimally contain an extracellular antigen recognition domain linked through a transmembrane domain to an intracellular activation domain or domains. Early CARs consisted of antibody single-chain variable fragments (scFvs) fused through a transmembrane domain to the cytoplasmic tail of the TCR signaling component CD3ζ. Second-generation CARs are those that incorporate a costimulatory domain membrane-proximal to the CD3ζ signaling domain, such as KYMRIAH®, YESCARTA®, and most of the clinically used CARs. Third- and fourth-generation CAR constructs are being developed, with each successive generation adding additional signaling capacity. Third-generation CARs contain two in-line costimulatory domains, whereas fourth-generation CAR T cells typically incorporate separate cytokine signals. Second-generation CARs differ in their choice of costimulatory domain, which affects the efficacy, response phenotype, and metabolic properties of the resulting CAR T cells. The most frequently used costimulatory domains derive from the CD28 family (CD28 and ICOS, Inducible T Cell Costimulator) and the tumor necrosis factor receptor (TNFR) family (4-1BB, CD27, and OX40). KYMRIAH® and YESCARTA® use the same scFv, which recognizes the B cell antigen CD19, but YESCARTA® incorporates a CD28-derived costimulatory domain, whereas KYMRIAH® incorporates a 4-1BB domain. T cells expressing these two different second-generation CARs have substantial and important functional differences, although the reasons for this are not entirely clear. CD28-based CARs seem to elicit stronger T cell activation as compared with 4-1BB-expressing CARs, tending toward an effector-like phenotype; with high interleukin-2 (IL-2) secretion and cytolytic capacity. However, in vivo persistence of CD28-based CARs is limited, and they are more prone to activation-induced cell death. In contrast, 4-1BB-based CAR T cells tend toward a central memory phenotype with slower effector response. 4-1BB CAR T cells are also more persistent, due to decreased exhaustion and up-regulation of BCL-2 family members, and have been found in vivo even years after treatment. It is likely that these strikingly distinct phenotypes arise from their activation of different downstream pathways.

CAR T cell therapy has transformed the care of refractory B cell malignancies and holds tremendous promise for many aggressive tumors. Despite remarkable efficacy in the treatment of some malignancies, CAR T cell therapy has several notable adverse reactions which can be life threatening. The most common severe reaction to CAR T cell therapy is the cytokine release syndrome (CRS). CRS occurs after the hundreds of millions of infused T cells release cytokines in a positive feedback loop, causing a systematic inflammatory response syndrome (SIRS). The SIRS reaction present in CRS can be clinically indistinguishable from sepsis and septic shock, with fevers, tachycardia, hypotension, and multiple organ system dysfunctions. Therefore, it is very important to reduce the frequency as well as the severity of CRS caused by CAR T cell therapy.

At the same time, some patients do not respond to anti-CD19 CAR T cell therapy. The reasons may be related to the patient's large tumor burden, the patient's T lymphocyte dysfunction, and the patient's complex immunosuppressive microenvironment. For B-Cell Acute Lymphoblastic Leukemia patients, there are high relapse rate, especially for high risk patients who has BCR-ABL gene fusions or TP53 mutations. Over 80% relapse rate has been reported for these high-risk patients. For non-Hodgkin's lymphoma (NHL), the current CD19 CAR-T therapy can only achieve less than 50% complete remission (CR) rate.

One of the important topics in CAR T cell research is whether the effectiveness of CAR T cell therapy can be improved by optimizing the structure of CAR. At present, optimization of CAR structural elements mainly focuses on the signaling elements of CAR, namely, the antigen recognition site and the costimulatory signaling domain. For example, the affinity of the antigen recognition region (e.g. scFv) for the target protein may be adjusted so that it can only recognize target antigen expressed on tumor cells, but not those expressed on normal cells so as to avoid on target off tumor effect. There are also results indicating CARs with different costimulatory domains would confer different biological effects. Compared to cells expressing CARs that contain the CD28 costimulatory domain, cells expressing CARs with the 4-1BB costimulatory domain tend to have slower effector response and longer persistent in the host. Researchers have also tried to optimize the three immunoreceptor tyrosine activation motifs (ITAMs) of CD3ζ, and it was found that CAR T cells with one mutated ITAM have stronger anti-tumor activity due to avoidance of cell death caused by over-activation.

Thus, there is a need for optimizing the structure of CAR to enhance the effectiveness of CAR T cell therapy.

### SUMMARY OF THE INVENTION

In one embodiment, the present disclosure provides an isolated chimeric antigen receptor (CAR) molecule comprising an antigen binding domain, a modified hinge and transmembrane domain (designated as HTM-1), a costimulatory domain, and an intracellular signaling domain, wherein the modified hinge and transmembrane domain (HTM-1) comprises the amino acid sequence of SEQ ID NO:2.

In one embodiment, the antigen binding domain of the CAR disclosed herein is a single chain antibody or single chain antibody fragment. In one embodiment, the antigen binding domain binds to a target antigen such as CD19. Other examples of target antigen include, but are not limited to, CD20, CD22, CD33, CD123, BCMA, CLL1, CD7, CS1, CEA, AFP, PSMA, GPC3, GD2, EGFRVIII, NKG2D, Mesothelin, Claudin 18.2, ROR3, and Muc1. One of ordinary skill in the art would readily incorporate into the CAR disclosed herein an antigen binding domain that would bind to any other target antigen of interest.

In one embodiment, the antigen binding domain of the CAR disclosed herein comprises a scFv that binds to CD19. In one embodiment, the anti-CD19 scFv comprises the amino acid sequences of SEQ ID NOs:4 and 6.

In one embodiment, the costimulatory domain of the CAR disclosed herein comprises a functional signaling domain of one of the following proteins: OX40, CD2, CD27, CD28, CDS, ICAM-1, LFA-1 (CD11a/CD18), ICOS (CD278), and 4-1BB (CD137).

In one embodiment, the intracellular signaling domain of the CAR disclosed herein comprises an intracellular signaling domain of CD3 zeta, or FcR gamma, or a functional fragment thereof. One of ordinary skill in the art would readily recognize that the intracellular signaling domain of the CAR disclosed herein may comprise any other suitable signaling domain known in the art.

In one embodiment, the present disclosure provides a nucleic acid construct comprising one or more nucleic acid sequences that encode the CAR disclosed herein, or fragments thereof. In another embodiment, there is provided an expression vector comprising the above nucleic acid construct. In another embodiment, there is provided a cell comprising the above expression vector. In another embodiment, there is provided a cell comprising the CAR disclosed herein. In another embodiment, there is provided a composition comprising the cell described above and a pharmaceutically acceptable carrier. In one embodiment, the cell is an immune cell. In one embodiment, the immune cell is a T cell.

In one embodiment, the present disclosure also provides a method of using the cells (e.g. T cells) expressing the CAR disclosed herein to treat cancer. In another embodiment, the present disclosure provides a method of using the cells (e.g. T cells) expressing the CAR disclosed herein to treat autoimmune diseases.

These and other aspects of the invention will be appreciated from the ensuing descriptions of the figures and detailed description of the invention.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-1E show the results of in vitro cytotoxic assays with T cells expressing conventional anti-CD19 CAR or HTM-1 CAR as effector cells against Nalm6 tumor cells as the target cells at the E:T ratio of 0.625:1 (FIG. 1A), 1.25:1 (FIG. 1B), 2.5:1 (FIG. 1C), 5:1 (FIG. 1D) and 10:1 (FIG. 1E). The trapezoid box in each figure represents the % of dead Nalm6 cells.

FIGS. 2A-2E show the results of in vitro cytotoxic assays with T cells expressing CD8-GG CAR or HTM-1 CAR as effector cells against Nalm6 tumor cells as the target cells at the E:T ratio of 0.625:1 (FIG. 2A), 1.25:1 (FIG. 2B), 2.5:1 (FIG. 2C), 5:1 (FIG. 2D) and 10:1 (FIG. 2E). The trapezoid box in each figure represents the % of dead Nalm6 cells.

### DETAILED DESCRIPTION OF THE INVENTION

The present specification discloses a new structure for a chimeric antigen receptor (CAR) comprising a modified CD8 hinge and transmembrane domain (HTM-1) having the amino acid sequence of SEQ ID NO:2. The modified CD8 hinge and transmembrane domain disclosed herein is expected to reduce the flexibility of the CAR, thus enhancing the functions of the CAR. In some embodiments, effector cells (e.g. T cells) expressing CAR with the modified CD8 hinge and transmembrane domain disclosed herein are expected to induce less release of pro-inflammatory cytokines.

In one embodiment, the CAR disclosed herein comprises an antigen binding domain, a modified CD8 hinge and transmembrane domain as disclosed herein, a costimulatory domain, and an intracellular signaling domain. One of ordinary skill in the art would readily select and construct an antigen binding domain, a costimulatory domain, and an intracellular signaling domain to be incorporated in the CAR disclosed herein. These various domains will be further discussed below.

In one embodiment, the present disclosure provides a new type of CAR that can specifically kill tumor cells expressing CD19. T cells expressing the new CAR are expected to more effectively control tumor burden in a host, and cause less secretion of pro-inflammatory cytokines. In one embodiment, the CAR disclosed herein comprises an anti-CD19 scFv, in addition to the modified CD8 hinge and transmembrane domain disclosed herein.

The present disclosure relates to polypeptides of CAR comprising the modified CD8 hinge and transmembrane domain disclosed herein and polynucleotides encoding the same. The present disclosure also provides vectors (e.g., viral vectors) comprising such polynucleotides and compositions comprising such vectors. The present disclosure also provides polynucleotides encoding the CAR disclosed herein and compositions comprising such polynucleotides. The present disclosure additionally provides engineered cells (e.g., T cells) comprising such polynucleotides and/or transduced with such vectors and compositions. In one embodiment, the present disclosure provides compositions (e.g., pharmaceutical compositions) including a plurality of such engineered T cells expressing CAR with the modified CD8 hinge and transmembrane domain disclosed herein. The present disclosure also provides methods for manufacturing such engineered T cells and compositions and uses (e.g., in treating a B cell lymphoma) of such engineered T cells and compositions. In another embodiment, the present disclosure provides a method of inducing immunity against a tumor comprising administering to a subject an effective amount of a cell comprising a polynucleotide, a vector, or a polypeptide of the present disclosure. In another embodiment, the present disclosure relates to cells comprising CAR with the modified CD8 hinge and transmembrane domain disclosed herein and their use in a T cell therapy, e.g., an autologous cell therapy for the treatment of a patient suffering from a cancer.

As used herein, the terms “comprise”, “comprises”, “comprising”, “includes”, “including”, “having” and their conjugates mean “including but not limited to”. As used herein, the singular form “a”, “an” and “the” include plural references unless the context clearly dictates otherwise.

Throughout this application, various embodiments of the present disclosure may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting. Each literature reference or other citation referred to herein is incorporated herein by reference in its entirety.

As used herein, an “antigen binding molecule,” “antigen binding domain,” or “antibody fragment” refers to any molecule that comprises the antigen binding regions (e.g., CDRs) of the antibody from which the molecule is derived. An antigen binding domain can include the antigenic complementarity determining regions (CDRs) that can be readily determined by one of ordinary skill in the art. Examples of antibody fragments include, but are not limited to, Fab, Fab′, F(ab′)2, Fv fragments, dAb, linear antibodies, scFv, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyper-proliferative disease or to a viral or bacterial antigen. In certain embodiments, the antigen binding molecule binds to CD19. In further embodiments, the antigen binding molecule is an antibody fragment that specifically binds to the antigen, including one or more of the complementarity determining regions (CDRs) thereof.

In some embodiments, the antigen binding molecule is a single chain variable fragment (scFv) as it is generally known in the art. A scFv polypeptide molecule is a covalently linked V_{H}-V_{L} heterodimer, which can be expressed from a gene fusion including V_{H}- and V_{L}-encoding genes linked by linker. The linker peptide (e.g., of about ten to about 25 amino acids) is usually rich in glycine for flexibility, as well as serine or threonine for solubility. The linker may either connect the N-terminus of the VH with the C-terminus of the VL or connect the C-terminus of the VH with the N-terminus of the VL. This protein retains the specificity of the original immunoglobulin, despite removal of the constant regions and the introduction of the linker. An scFv may also include an N-terminal peptide sequence, which sometimes is referred to as a “signal peptide” or “leader sequence”. A number of methods are available to one of ordinary skill in the art to construct a scFv from the light and heavy chain variable regions of an antibody, see e.g., U.S. Pat. Nos. 5,091,513; 5,132,405; and 4,946,778.

The terms “VL”, “VL region”, and “VL domain” are used interchangeably to refer to the light chain variable region of an antigen binding domain such as an antibody or an antigen-binding fragment thereof, and comprises one, two, or three CDRs.

The terms “VH”, “VH region”, and “VH domain” are used interchangeably to refer to the heavy chain variable region of an antigen binding domain such as an antibody or an antigen-binding fragment thereof, and comprise one, two, or three CDRs.

As it is generally known in the art, there are a number of commonly used definitions of CDRs, such as Kabat numbering, Chothia numbering, AbM numbering, or contact numbering.

As used herein, the terms “genetic engineering” or “engineering” are used interchangeably and mean a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which may either be obtained from a patient or a donor. The cell may be modified to express an exogenous construct, such as a construct encoding a CAR comprising the modified CD8 hinge and transmembrane domain disclosed herein, which is incorporated into the cell's genome.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods of using the CAR disclosed herein include, but are not limited to, cancers of the immune system including lymphoma, leukemia, myeloma, and other leukocyte malignancies.

In some embodiments, cancers that can be treated by the methods of using the CAR disclosed herein include, but are not limited to, B-cell lymphomas, acute lymphoblastic leukemia (ALL), AIDS-related lymphoma, ALK-positive large B-cell lymphoma, Burkitt's lymphoma, chronic lymphocytic leukemia, (CLL), classical Hodgkin lymphoma, diffuse large B-cell lymphoma (DLBCL), follicular lymphoma, intravascular large B-cell lymphoma, large B-cell lymphoma arising in HHV8-associated multicentric Castleman's disease, lymphomatoid granulomatosis, lymphoplasmacytic lymphoma, mantle cell lymphoma (MCL), marginal zone B-cell lymphoma (MZL), mucosa-associated lymphatic tissue lymphoma (MALT), nodal marginal zone B cell lymphoma (NMZL), nodular lymphocyte predominant Hodgkin's lymphoma, non-Hodgkin's lymphoma, plasmablastic lymphoma, primary central nervous system lymphoma, primary effusion lymphoma, splenic marginal zone lymphoma (SMZL), and Waldenstrom's macroglobulinemia, or a combination thereof.

An “anti-tumor effect” as used herein refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

As used herein, the term “autoimmune disease” encompasses diseases or conditions such as achalasia, amyloidosis, ankylosing spondylitis, anti-gbm/anti-tbm nephritis, antiphospholipid syndrome, arthritis, autoimmune angioedema, autoimmune encephalomyelitis, autoimmune hepatitis, autoimmune myocarditis, autoimmune oophoritis, autoimmune orchitis, autoimmune pancreatitis, autoimmune retinopathy, autoimmune urticaria, Behcet's disease, celiac disease, chagas disease, chronic inflammatory demyelinating polyneuropathy, Cogan's syndrome, congenital heart block, Crohn's disease, dermatitis, dermatomyositis, discoid lupus, Dressler's syndrome, endometriosis, fibromyalgia, fibrosing alveolitis, granulomatosis with polyangiitis, Graves' disease, Guillain-Barre syndrome, herpes gestationis, immune thrombocytopenic purpura, interstitial cystitis, juvenile arthritis, juvenile diabetes (type 1 diabetes), juvenile myositis, Kawasaki disease, Lambert-Eaton syndrome, lichen planus, lupus, Lyme disease, multiple sclerosis, myasthenia gravis, myositis, neonatal lupus, neutropenia, palindromic rheumatism, peripheral neuropathy, polyarteritis nodosa, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, reactive arthritis, retroperitoneal fibrosis, rheumatic fever, rheumatoid arthritis, sarcoidosis, Schmidt syndrome, scleritis, scleroderma, Sjögren's syndrome, thrombocytopenic purpura, type 1 diabetes, ulcerative colitis, uveitis, vasculitis, or vitiligo.

A “therapeutically effective amount,” “effective dose,” “effective amount,” or “therapeutically effective dosage” of a therapeutic agent, e.g., engineered T cells expressing the CAR disclosed herein, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission.

### Chimeric Antigen Receptors (CAR)

As it is generally known in the art, chimeric antigen receptors are genetically engineered receptors comprising at least an extracellular antigen binding domain, a hinge domain, a transmembrane domain and a cytoplasmic signaling domain. These engineered receptors can be readily inserted into and expressed by immune cells, such as T cells in accordance with techniques known in the art. With a CAR, a single receptor can be programmed to both recognize a specific antigen and, when bound to that antigen, activates the immune cell to attack and destroy the cell bearing that antigen. When these antigens exist on tumor cells, an immune cell that expresses the CAR can target and kill the tumor cell.

In some embodiments, the present disclosure relates to CARs comprising a modified hinge and transmembrane domain comprising the amino acid sequence of SEQ ID NO:2.

As it is generally known in the art, a CAR is a modular structure comprising multiple domains or regions, and one of ordinary skill in the art would readily construct a CAR comprising the required domains or regions generally known in the art. In some embodiments, the CAR disclosed herein comprises the following domains:
<First signal peptide> <VH of a scFv> <scFv Linker> <VL of a scFv> <hinge and transmembrane domain> <4-1BB costimulatory domain> <CD3ζ signaling domain>.

In one embodiment, the first signal peptide comprises the amino acid sequence of SEQ ID NO:10, encoded by a polynucleotide comprising the sequence of SEQ ID NO:9. In one embodiment, the scFv linker comprises the amino acid sequence of SEQ ID NO:8, encoded by a polynucleotide comprising the sequence of SEQ ID NO:7. In one embodiment, the hinge and transmembrane domain comprises the amino acid sequence of SEQ ID NO:2, encoded by a polynucleotide comprising the sequence of SEQ ID NO:1. In one embodiment, the 4-1BB and CD3ζ domain comprises the amino acid sequence of SEQ ID NO:12, encoded by a polynucleotide comprising the sequence of SEQ ID NO:11.

In some embodiments, the CARs disclosed herein comprise an antigen binding domain, such as a scFv, that specifically binds to a tumor-associated antigen, e.g. human CD19. In other embodiments, the present disclosure also relates to engineered T cells expressing such CARs.

As used herein, the term “tumor associated antigen” (TAA) encompasses a molecule or a portion thereof, which is displayed on the surface of a cell or a molecule which is present within the milieu of a tumor, that is within the tumor micro-environment (TME). In some embodiments, a TAA encompasses a cell surface tumor associated antigen (TAA). In some embodiments, the cell is a tumor cell. In some embodiments, the cell is a non-tumor cell present in the milieu of a tumor, for example but not limited to a cell present within vasculature tissue associated with a tumor or cancer. In some embodiments, a TAA is an angiogenic antigen in a tumor micro-environment. In some embodiments, a TAA is an antigen on a blood vessel in a tumor micro-environment. In some embodiments, the cell is a stromal cells present in the milieu of a tumor. In some embodiments, a TAA is a stromal cell antigen within a tumor micro-environment. In some embodiments, a TAA encompasses an extracellular epitope of a tumor-cell-surface antigen. In some embodiments, a TAA encompasses an extracellular matrix antigen.

In some embodiments, a TAA comprises an antigen present in a TME. In some embodiments, a TAA comprises a molecule secreted by a tumor cell into the TME. In some embodiments, a TAA comprises an effector molecule secreted by a tumor cell into the TME. In some embodiments, a TAA comprises an effector molecule secreted by a tumor cell into the TME in order to downregulate or inhibit the activity of cytotoxic natural killer (NK) or T cells. In some embodiments, a TAA comprises soluble activating receptor ligand secreted by a tumor cell into the TME in order to block the recognition of the tumor cell by a NK cell or T cell. In some embodiments, a TAA comprises a suppressive immune cell in the TME that would otherwise inhibit NK cell activation.

In some embodiments, the tumor associated antigen (TAA) is a tumor antigen. In some embodiments, tumor antigens comprise those antigens are presented on tumor cells. In some embodiments, the tumor antigen is present on a cell of solid tumor. In some embodiments, the tumor antigen is a cancer antigen, present on a cell of a non-solid tumor.

In some embodiments, the solid tumor comprises a sarcoma or a carcinoma, a fibrosarcoma, a myxosarcoma, a liposarcoma, a chondrosarcoma, an osteogenic sarcoma, a chordoma, an angiosarcoma, an endotheliosarcoma, a lymphangiosarcoma, a lymphangioendotheliosarcoma, a synovioma, a mesothelioma, an Ewing's tumor, a leiomyosarcoma, a rhabdomyosarcoma, a colon carcinoma, a pancreatic cancer or tumor, a breast cancer or tumor, an ovarian cancer or tumor, a prostate cancer or tumor, a squamous cell carcinoma, a basal cell carcinoma, an adenocarcinoma, a sweat gland carcinoma, a sebaceous gland carcinoma, a papillary carcinoma, a papillary adenocarcinomas, a cystadenocarcinoma, a medullary carcinoma, a bronchogenic carcinoma, a renal cell carcinoma, a hepatoma, a bile duct carcinoma, a choriocarcinoma, a seminoma, an embryonal carcinoma, a Wilm's tumor, a cervical cancer or tumor, a uterine cancer or tumor, a testicular cancer or tumor, a lung carcinoma, a small cell lung carcinoma, a bladder carcinoma, an epithelial carcinoma, a glioma, an astrocytoma, a medulloblastoma, a craniopharyngioma, an ependymoma, a pinealoma, a hemangioblastoma, an acoustic neuroma, an oligodenroglioma, a schwannoma, a meningioma, a melanoma, a neuroblastoma, or a retinoblastoma. In some embodiments, the solid tumor comprises an adrenocortical tumor (adenoma and carcinoma), a colorectal carcinoma, a desmoid tumor, a desmoplastic small round cell tumor, an endocrine tumor, an Ewing sarcoma, a germ cell tumor, a hepatoblastoma a hepatocellular carcinoma, an osteosarcoma, a soft tissue sarcoma other than rhabdomyosarcoma, and a Wilms tumor.

In one embodiment, the TAA can be, but is not limited to, CD19, 5T4, ROR1, EGFR, carcinoembryonic antigen (CEA, such as CEACAM5, CEACAM6), FcγRI, FcγRIIa FcγRIIb FcγRIIIa FcγRIIIb, CD28, CD137, CTLA-4, FAS, FAP (Fibroblast activation protein), LGR5, C5aR1, A2AR, fibroblast growth factor receptor 1 (FGFR1), FGFR2, FGFR3, FGFR4, glucocorticoid-induced TNFR-related (GITR) protein, lymphotoxin-beta receptor (LTβR), toll-like receptors (TLR), tumor necrosis factor-related apoptosis-inducing ligand-receptor 1 (TRAIL receptor 1), TRAIL receptor 2, prostate-specific membrane antigen (PSMA) protein, prostate stem cell antigen (PSCA) protein, tumor-associated protein carbonic anhydrase IX (CAIX), epidermal growth factor receptor 1 (EGFR1), EGFRvIII, human epidermal growth factor receptor 2 (Her2/neu; Erb2), ErbB3 (HER3), Folate receptor, ephrin receptors, PDGFRa, ErbB-2, CD20, CD22, CD30, CD33, CD40, CD37, CD38, CD70, CD74, CD56, CD40), CD80, CD86, CD2, p53, cMet (tyrosine-protein kinase Met) (hepatocyte growth factor receptor) (HGFR), MAGE-A1, MAGE-A2, MAGE-A3, MAGE-A4, MAGE-A6, MAGE-A10, MAGE-A12, BAGE, DAM-6, DAM-10, GAGE-1, GAGE-2, GAGE-8, GAGE-3, GAGE-4, GAGE-5, GAGE-6, GAGE-7B, NA88-A, NY-ESO-1, BRCA1, BRCA2, MART-1, MC1R, Gp100, PSA, PSM, Tyrosinase, Wilms' tumor antigen (WT1), TRP-1, TRP-2, ART-4, CAMEL, Cyp-B, hTERT, hTRT, iCE, MUC1, MUC2, P-cadherin, Myostatin (GDF8), Cripto (TDGF1), MUC5AC, PRAME, P15, RU1, RU2, SART-1, SART-3, WT1, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, ETV6/AML, LDLR/FUT, Pml/RARα, TEL/AML1, CD28, CD137, CanAg, Mesothelin, DR5, PD-1, PD1L, IGF-1R, CXCR4, Neuropilin 1, Glypicans, EphA2, CD138, B7-H3, B7-H4, gpA33, GPC3, SSTR2, or VEGF-R2.

In one embodiment, the antigen binding domain of the CAR disclosed herein binds to CD19. In one embodiment, the anti-CD19 antigen binding domain comprises a scFv, and the scFv comprises the amino acid sequences of SEQ ID NOs:4 and 6.

### Costimulatory Domains

As it is known in the art, the costimulatory domain of a CAR is designed to provide costimulatory signaling to an activating domain, which then activates at least one of the normal effector functions of the immune cell. Effector function of a T cell, for example, can be cytolytic activity or helper activity including the secretion of cytokines.

In certain embodiments, suitable costimulatory domains include, but are not limited to, the costimulatory domain of 4-1BB/CD137, activating NK cell receptors, an immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CDS, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that specifically binds with CD83, LIGHT, LIGHT, LTBR, Ly9 (CD229), Ly108), lymphocyte function-associated antigen-1 (LFA-1; CD1-la/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof. One of ordinary skill in the art would readily recognize and determine a costimulatory domain from these proteins. The polypeptide sequences of these costimulatory domains, as well as the polynucleotide sequences encoding the same, are well-known in the art.

In some embodiments, the polypeptide sequence of a costimulatory domain comprises a polypeptide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to the polypeptide sequence of which is known in the art. In some embodiments, the polynucleotide encoding a costimulatory domain comprises a nucleotide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to the nucleotide sequence of which is known in the art.

### Intracellular Signaling/Activating Domains

Intracellular signaling or activating domains that can be incorporated into a CAR is generally known in the art. For example, CD3 is an element of the T cell receptor on native T cells, and has been shown to be an important intracellular activating element in CARs. In some embodiments, the CD3 is CD3-zeta or CD3-epsilon, the polynucleotide and polypeptide sequences of each of which are well-known in the art.

In some embodiments, the polypeptide sequence of an intracellular signaling or activating domain comprises a polypeptide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to the polypeptide sequence of which is known in the art. In some embodiments, the polynucleotide encoding an intracellular signaling or activating domain comprises a nucleotide sequence at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to the nucleotide sequence of which is known in the art.

### Leader Peptides or Leader Sequences

In some embodiments, the CAR disclosed herein may further comprise a leader peptide (also referred to herein as a “signal peptide” or “leader sequence”). Leader peptides suitable for incorporation in CAR are well-known in the art. In certain embodiments, the leader peptide comprises an amino acid sequence that is at least about 75%, at least about 80%, at least about 85%, at least about 90%, at least about 95%, at least about 96%, at least about 97%, at least about 98%, at least about 99%, or 100% identical to the amino acid sequence of leader sequences known in the art.

### Vectors, Cells, and Pharmaceutical Compositions

In another embodiment, provided herein are nucleic acid constructs comprising one or more nucleic acid sequences that encode the CAR disclosed herein, or fragments thereof. In another embodiment, provided herein are expression vectors comprising the above nucleic acid constructs.

Any vector known in the art is suitable for the present invention. In some embodiments, the vector is a viral vector. In some embodiments, the vector can be, but is not limited to, a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof.

In other aspects, provided herein are cells comprising the nucleic acid constructs or vectors described above. In some embodiments, the present disclosure provides cells comprising a polynucleotide encoding the CAR described herein. In other embodiments, the present disclosure provides cells comprising the CAR described herein.

Any cell may be used as a host cell for the polynucleotides, the vectors, or the polypeptides of the present disclosure. In some embodiments, the cell can be a prokaryotic cell, fungal cell, yeast cell, or higher eukaryotic cells such as a mammalian cell. Suitable prokaryotic cells generally known in the art include, without limitation, eubacteria, such as Gram-negative or Gram-positive bacteria. In some embodiments, the cell is a human cell. In some embodiments, the cell is an immune cell. In some embodiments, the immune cell can be a T cell, a B cell, a tumor infiltrating lymphocyte (TIL), a TCR expressing cell, a natural killer (NK) cell, a dendritic cell, a granulocyte, an innate lymphoid cell, a megakaryocyte, a monocyte, a macrophage, a platelet, a thymocyte, or a myeloid cell. In one embodiment, the immune cell is an allogeneic T cell, a heterologous T cell, or any combination thereof.

The cell of the present invention may be obtained through any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, or tumors. In addition, the T cells can be derived from one or more T cell lines available in the art. Methods for isolating T cells for T cell therapy are generally known in the art, e.g. in U.S. Patent Publication No. 2013/0287748, which is herein incorporated by references in its entirety.

Pharmaceutical compositions of the present disclosure may comprise a cell expressing the CAR disclosed herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present invention are in one aspect formulated for intravenous administration.

Pharmaceutical compositions of the present disclosure may be administered in a manner appropriate to the disease to be treated (or prevented). The quantity and frequency of administration are determined by such factors as the condition of the patient, and the type and severity of the patient's disease according to practices generally known in the art.

### Uses of the CAR

Another aspect of the disclosure is directed to a method of making a cell expressing the CAR disclosed herein. In some embodiments, the method comprises transducing a cell with a polynucleotide encoding the CAR comprising the modified CD8 hinge and transmembrane domain disclosed herein. In some embodiments, the method comprises transducing a cell with a vector comprising polynucleotide sequences encoding the CAR disclosed herein.

Another aspect of the present disclosure is directed to a method of inducing immune responses against a tumor comprising administering to a subject an effective amount of cells comprising the polynucleotide encoding a CAR comprising the modified CD8 hinge and transmembrane domain described herein, the vector described herein, or the CAR described herein. In one embodiment, the method comprises administering to a subject an effective amount of cells comprising a polynucleotide encoding the CAR disclosed herein. In another embodiment, the method comprises administering to a subject an effective amount of cells comprising a vector comprising polynucleotide sequences encoding the CAR disclosed herein. In another embodiment, the method comprises administering to a subject an effective amount of cells comprising the CAR disclosed herein. An “immune response” refers to the action of a cell of the immune system (for example, T lymphocytes, B lymphocytes, natural killer (NK) cells, macrophages, eosinophils, mast cells, dendritic cells or neutrophils) and soluble macromolecules produced by any of these cells (for example antibodies, cytokines, and complement) that results in selective targeting, binding to, damage to, destruction of, and/or elimination from a vertebrate's body of invading pathogens, cells or tissues infected with pathogens, cancerous or other abnormal cells.

Another aspect of the present disclosure is directed to a method of using the CAR disclosed herein to treat a cancer in a subject in need thereof. In one embodiment, the method comprises administering to the subject polynucleotide sequences encoding the CAR disclosed herein. In another embodiment, the method comprises administering a vector comprising polynucleotide sequences encoding the CAR disclosed herein. In another embodiment, the method comprises administering to the subject cells (e.g. T cells) expressing the CAR disclosed herein. In one embodiment, T cells can be isolated from the subject (or from a third party) and be engineered to express the CAR disclosed herein according to commonly used techniques in the art.

Another aspect of the present disclosure is directed to a method of using the CAR disclosed herein to treat an autoimmune disease in a subject in need thereof. In one embodiment, the method comprises administering to the subject polynucleotide sequences encoding the CAR disclosed herein. In another embodiment, the method comprises administering a vector comprising polynucleotide sequences encoding the CAR disclosed herein. In another embodiment, the method comprises administering to the subject cells (e.g. T cells) expressing the CAR disclosed herein. In one embodiment, T cells can be isolated from the subject (or from a third party) and be engineered to express the CAR disclosed herein according to commonly used techniques in the art.

In some embodiments, T cells engineered to express a CAR comprising the modified CD8 hinge and transmembrane domain disclosed herein can be administered to a subject or patient at a therapeutically effective amount. For example, a therapeutically effective amount of the T cells can be at least about 10⁴ cells, at least about 10⁵ cells, at least about 10⁶ cells, at least about 10⁷ cells, at least about 10⁸ cells, at least about 10⁹, or at least about 10¹⁰. In some embodiments, the therapeutically effective amount of the engineered CAR T cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

The methods described above can be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In certain embodiments, the methods induce a complete response. In other embodiments, the methods induce a partial response.

In some embodiments, the methods described above may further comprise administering a second therapeutic. Examples for the second therapeutic include, but are not limited to, a chemotherapeutic agent, a radioactive therapeutic agent, a cytokine, or a cytokine inhibitor. In some embodiments, compositions comprising cells expressing the CAR disclosed herein, and the second therapeutic are administered each in an amount effective to treat the disease or condition in the subject. In certain embodiments, compositions comprising cells expressing the CAR disclosed herein may be administered prior to, in conjunction with, and/or subsequent to the administration of the second therapeutic.

In the description presented herein, each of the steps of the invention and variations thereof are described. This description is not intended to be limiting and changes in the components, sequence of steps, and other variations would be understood to be within the scope of the present invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Example 1

CAR with Modified Hinge and Transmembrane Domain

### Materials and Methods

### Cell Lines and Cell Culture Conditions

Cell lines were cultured according to the manufacturers' recommendations. NALM-6 is a pre-B cell acute lymphoblastic leukemia (ALL) cell line with high expression of CD19 (German DSMZ cell collection Cat #: ACC128). NALM-6-GFP-luciferase (luc) is a stable cell line engineered to express GFP-luciferase. The method of tumor cells culture is described in Zhang et al. Secretion of Human Soluble Programmed Cell Death Protein 1 by Chimeric Antigen Receptor-Modified T Cells Enhances Anti-Tumor Efficacy. Cytotherapy (2020) 22 (12):734-43.

### Generation of CAR Constructs

Generation of lentiviral constructs and production of lentiviral particles are described in Kutner et al. Production, Concentration and Titration of Pseudotyped HIV-1-Based Lentiviral Vectors. Nat Protoc (2009) 4(4):495-505. The conventional second-generation 2nd CAR was constructed by the fusion of CD19 scFv, CD8 hinge and transmembrane, 4-1BB, and CD3z. The structure of CAR with the modified CD8 hinge region (CD8-GG) is same to the 2nd CAR except for deletion of two consecutive glycine in the CD8 hinge as disclosed in U.S. patent applications Nos. 63/272,617 and 63/320,930.

### Cytotoxicity Assay

Target cells NALM6 stably expressing GFP (NALM6-GFP) were incubated at the indicated ratios with CAR T cells for 4 hours. The cells were then harvested, and 7-AAD was added prior to flow cytometric analysis. The residual live target cells were GFP+7 AAD−. Apoptotic target cells were GFP+7 AAD+.

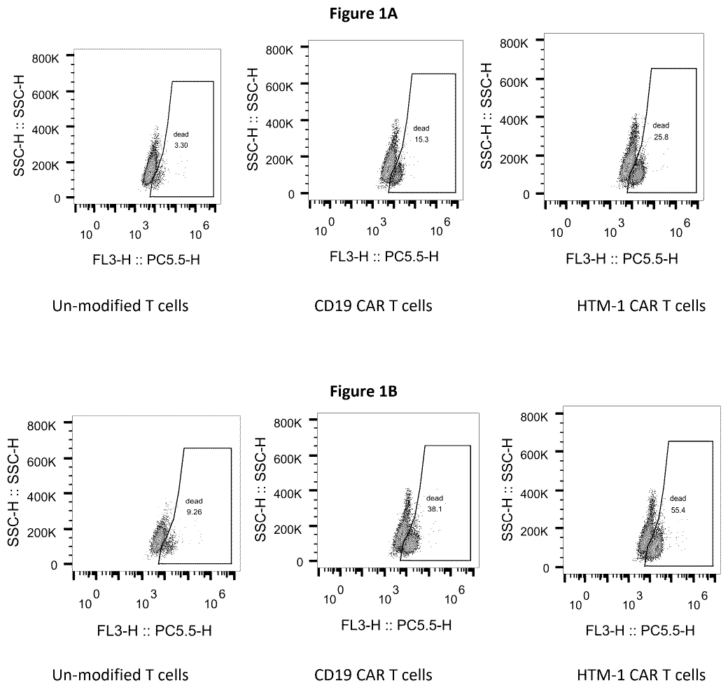

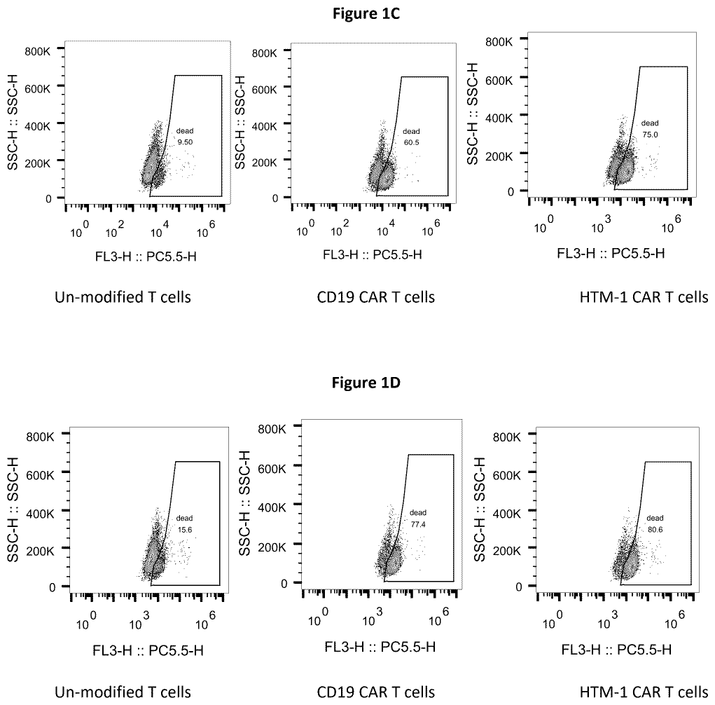

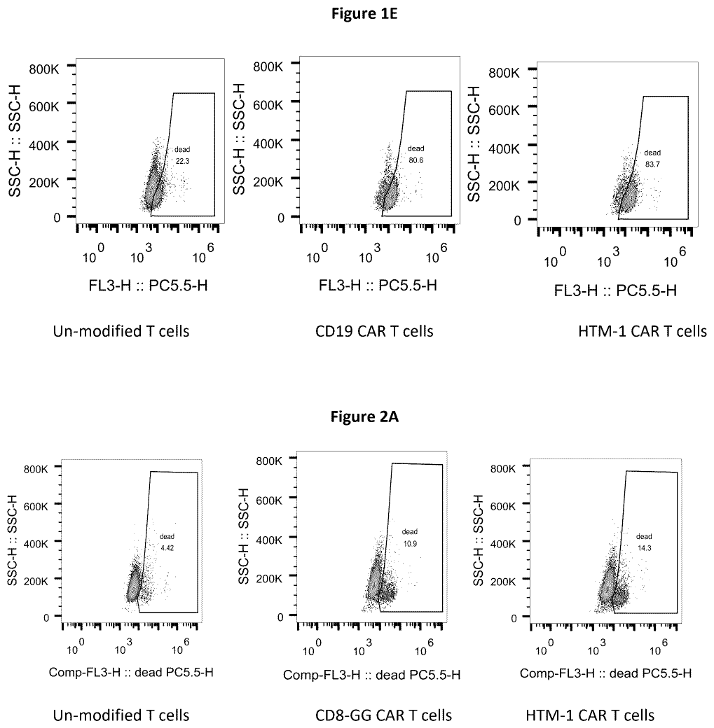

FIGS. 1A-1E show the cytotoxic functions of T cells expressing conventional anti-CD19 CAR or CAR comprising the HTM-1 domain disclosed herein. The conventional anti-CD19 CAR was constructed by the fusion of CD19 scFv, CD8 hinge and transmembrane, 4-1BB, and CD3z domains as published in the art. The results show that T cells expressing CAR with the HTM-1 domain have higher cytotoxic activities as compared to T cells expressing the conventional anti-CD19 CAR.

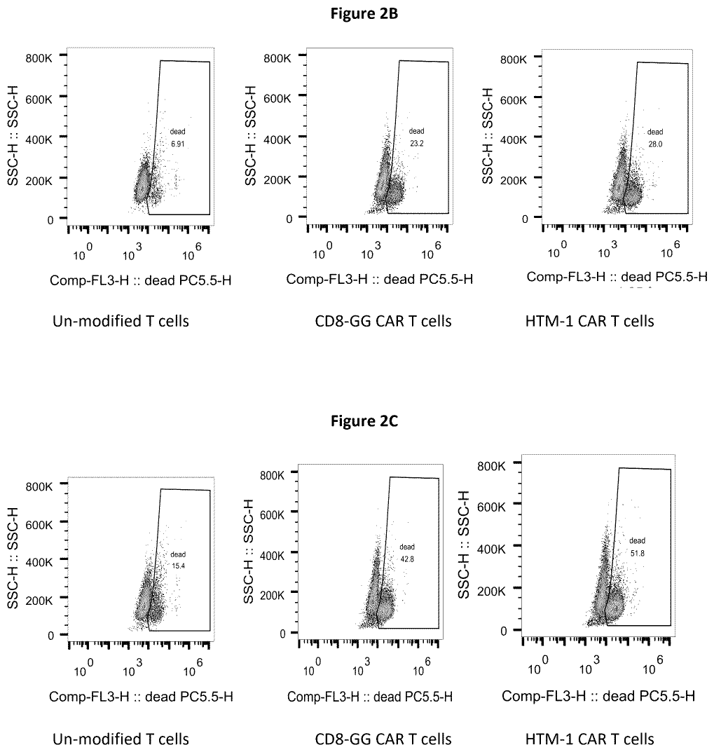

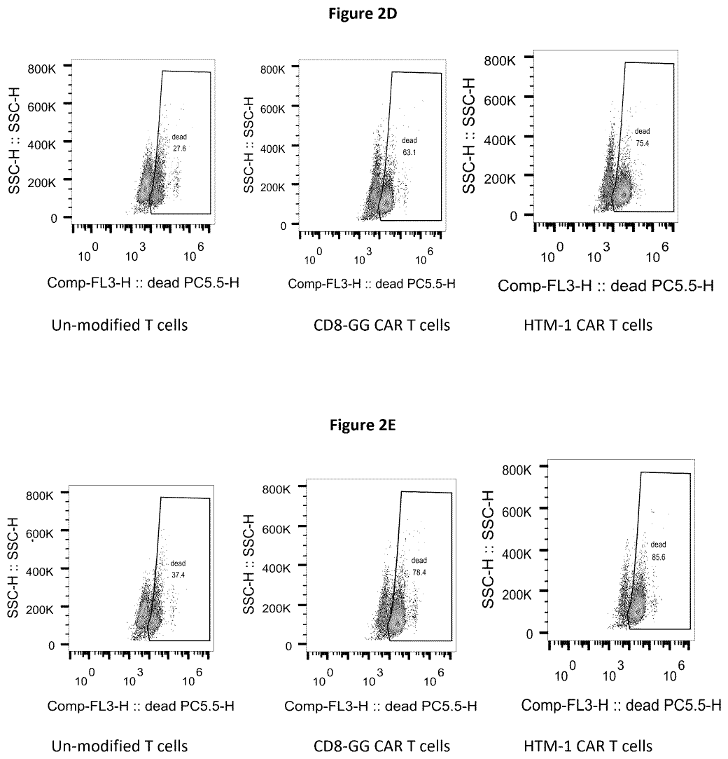

FIGS. 2A-2E show the cytotoxic activities of T cells expressing CAR with the HTM-1 domain as compared to T cells expressing CAR with a modified CD8 hinge region (CD8-GG as disclosed in U.S. patent applications Nos. 63/272,617 and 63/320,930). The results show that T cells expressing CAR with the HTM-1 domain have higher cytotoxic activities as compared to T cells expressing CAR with the CD8-GG domain.

In one embodiment of the present CAR, comprising from the amino to the carboxyl terminus, an antigen binding domain, a modified hinge and transmembrane domain (HTM-1), a costimulatory domain, and an intracellular signaling domain. Examples of the various components of the CAR have been described above. In one embodiment, the CAR comprises from the amino to the carboxyl terminus an anti-CD19 antigen binding domain (e.g. a scFv comprising the amino acid sequences of SEQ ID NOs:4 and 6), a modified CD8 hinge and transmembrane domain (SEQ ID NO:2), a 4-1BB costimulatory domain and a CD3ζ signaling domain (SEQ ID NO:12). The table below lists examples of the amino acid and nucleic acid sequences for the various components of the CAR:

| Description | Sequences |
|---|---|
| Modified CD8 | GCGGCCGCATTCGTGCCGGTCTTCCTGCCAGCGAAGCCCACCACGA |
| hinge and | CGCCAGCGCCGCGACCACCAACACCGGCGCCCACCATCGCGTCGCA |
| transmembrane | GCCCCTGTCCCTGCGCCCAGAGGCGTGCCGGCCAGCGGCGGCAGTG |
| domain | CACACGAGGGGGCTGGACTTCGCCTGTGATATCTACATCTGGGCGC |
| (SEQ ID NO: 1) | CCTTGGCCGGGACTTGTGGGGTCCTTCTCCTGTCACTGGTTATCAC |
| | CCTTTACTGCAACCACAGGAAC |
| Modified CD8 | AAAFVPVFLPAKPTTTPAPRPPTPAPTIASQPLSLRPEACRPAAAV |
| hinge and | HTRGLDFACDIYIWAPLAGTCGVLLLSLVITLYCNHRN |
| transmembrane | In one embodiment, the sequence AAA at the |
| domain | beginning functions as a linker. |
| (SEQ ID NO: 2) | |
| Anti-CD19 scFv | GATATTCAAATGACCCAGTCCCCGTCCTCCCTGAGTGCCTCCGTCG |
| VL | GTGACCGTGTTACGATTACCTGCCGTGCGAGCCAAGACATCTCTAA |
| (SEQ ID NO: 3) | ATACCTGAACTGGTATCAGCAAAAACCGGATCAGGCACCGAAACTG |
| | CTGATCAAACATACCTCACGTCTGCACTCGGGTGTGCCGAGCCGCT |
| | TTAGTGGTTCCGGCTCAGGTACCGATTACACCCTGACGATCAGCTC |
| | TCTGCAGCCGGAAGACTTTGCCACGTATTACTGCCAGCAAGGTAAT |
| | ACCCTGCCGTATACGTTCGGCCAAGGTACCAAACTGGAAATCAAA |
| Anti-CD19 scFv | DIQMTQSPSSLSASVGDRVTITCRASQDISKYLNWYQQKPDQAPKL |
| VL | LIKHTSRLHSGVPSRFSGSGSGTDYTLTISSLQPEDFATYYCQQGN |
| (SEQ ID NO: 4) | TLPYTFGQGTKLEIK |
| Anti-CD19 scFv | GAAGTGCAGCTGGTTGAAAGCGGTGGTGGTCTGGTTCAACCGGGTC |
| VH | GTTCCCTGCGTCTGTCATGTACGGCGAGTGGTGTCTCCCTGCCGGA |
| (SEQ ID NO: 5) | CTATGGCGTGTCCTGGATTCGTCAGCCGCCGGGTAAAGGCCTGGAA |
| | TGGATTGGTGTCATCTGGGGCAGTGAAACCACGTATTACAACTCGG |
| | CCCTGAAAAGCCGTTTCACCATCTCTCGCGATAACAGTAAAAATAC |
| | GCTGTACCTGCAGATGAATAGCCTGCGCGCGGAAGACACCGCCGTT |
| | TACTACTGCGCAAAACATTACTACTACGGTGGCAGCTATGCTATGG |
| | ATTACTGGGGTCAAGGCACGCTGGTCACCGTTTCGTCA |
| Anti-CD19 scFv | EVQLVESGGGLVQPGRSLRLSCTASGVSLPDYGVSWIRQPPGKGLE |
| VH | WIGVIWGSETTYYNSALKSRFTISRDNSKNTLYLQMNSLRAEDTAV |
| (SEQ ID NO: 6) | YYCAKHYYYGGSYAMDYWGQGTLVTVSS |
| scFv linker | GGTGGCGGTGGCTCGGGCGGTGGTGGGTCGGGTGGCGGCGGATCT |
| (SEQ ID NO: 7) | |
| scFv linker | GGGGSGGGGSGGGGS |
| (SEQ ID NO: 8) | |
| Signal peptide | ATGGCCTTACCAGTGACCGCCTTGCTCCTGCCGCTGGCCTTGCTGC |
| (SEQ ID NO: 9) | TCCACGCCGCCAGGCCG |
| Signal peptide | MALPVTALLLPLALLLHAARP |
| (SEQ ID NO: 10) | |
| 4-1BB, CD3z | AAACGGGGCAGAAAGAAACTCCTGTATATATTCAAACAACCATTTA |
| signaling domains | TGAGACCAGTACAAACTACTCAAGAGGAAGATGGCTGTAGCTGCCG |
| (SEQ ID NO: 11) | ATTTCCAGAAGAAGAAGAAGGAGGATGTGAACTGAGAGTGAAGTTC |
| | AGCAGGAGCGCAGACGCCCCCGCGTACAAGCAGGGCCAGAACCAGC |
| | TCTATAACGAGCTCAATCTAGGACGAAGAGAGGAGTACGATGTTTT |
| | GGACAAGAGACGTGGCCGGGACCCTGAGATGGGGGGAAAGCCGAGA |
| | AGGAAGAACCCTCAGGAAGGCCTGTACAATGAACTGCAGAAAGATA |
| | AGATGGCGGAGGCCTACAGTGAGATTGGGATGAAAGGCGAGCGCCG |
| | GAGGGGCAAGGGGCACGATGGCCTTTACCAGGGTCTCAGTACAGCC |
| | ACCAAGGACACCTACGACGCCCTTCACATGCAGGCCCTGCCCCCTC |
| | GC |
| 4-1BB, CD3z | KRGRKKLLYIFKQPFMRPVQTTQEEDGCSCRFPEEEEGGCELRVKF |
| signaling domains | SRSADAPAYKQGQNQLYNELNLGRREEYDVLDKRRGRDPEMGGKPR |
| (SEQ ID NO: 12) | RKNPQEGLYNELQKDKMAEAYSEIGMKGERRRGKGHDGLYQGLSTA |
| | TKDTYDALHMQALPPR |

### Example 2

Clinical Uses of CAR with Modified Hinge and Transmembrane Domain

The present example describes clinical results of treating B cell acute lymphocytic leukemia (B-ALL) patients with T cells expressing the CAR disclosed herein, i.e. CAR comprising an anti-CD19 antigen binding domain, the modified CD8 hinge and transmembrane domain (HTM-1 domain), a 4-1BB costimulatory domain and a CD3ζ signaling domain.

Lentivirus vectors with the CAR gene sequence were manufactured through a three-plasmid system, i.e. the pCDH-EF1-MCS, which is the main plasmid, the pMD2.G, which is the lentiviral packaging helper plasmid, and the psPAX2, which is the lentiviral packaging plasmid. The three plasmids were transiently transfected into 293T cells, and then secreted into the supernatant for collection and purification to form the final packaged vectors. The vectors were mixed with activated peripheral blood mononuclear cells (PBMCs) collected from the patient to transfect the CAR onto the CD3+ T cells contained in the PBMC. Finally, after 10 days of cultivation, T cells (part of which contains CAR) were transfused back to patients.

### Patient 1

Age 22; disease stage and molecular characterization: B-ALL, central nervous system leukemia, with the following genetic mutations: PRKDC, PLCG2, IL7R, NF1, KMT2D; fusion gene: WT1; express CD19.

Pre-treatment lymphodepletion: Fludarabine 50 mg for 3 days, and cyclophosphamide 0.5 g for 3 days.

Dosage: 1.17×10⁸ CD3+ T cells, autologous, with 59.8% CAR T cells. Adverse event: none. Anti-tumor response: complete remission up to 64 days after treatment.

### Patient 2

Age 53; disease stage and molecular characterization: B Philadelphia chromosome-like acute lymphoblastic leukemia (or called Ph-like positive B-ALL), relapsed from previous hematopoietic stem cell transplantation (HSCT). Bone marrow morphology: primitive naïve lymphocytes account for 91.5%. Minimal residual disease (MRD): 87.13% malignant naïve B lymphocytes; MRD expresses CD19 (64.27%) prior to CAR-T infusion.

Pre-treatment lymphodepletion: Fludarabine 50 mg for 3 days, and cyclophosphamide 0.6 g for 3 days.

Dosage: 1.47×10⁸ CD3+ T cells, allogenic from the HSCT donor, with 51.4% CAR T cells. Adverse event: CRS. Anti-tumor response: complete remission up to 60 days after treatment.

### Patient 3

Age 14; disease stage and molecular characterization: B-ALL with AP3B1 mutation and WT1 fusion gene. Immunophenotyping: 89% of total nuclear cells are abnormal lymphocytes. MRD expresses CD19.

Pre-treatment lymphodepletion: Fludarabine 41 mg for 3 days, and cyclophosphamide 0.4 g for 3 days.

Dosage: 1.24×10⁸ CD3+ T cells, autologous, with 36.2% CAR T cells. Adverse event: none. Anti-tumor response: complete remission up to 40 days after treatment.

Summary. All three patients in this example achieved complete remission (CR) after the infusion of CAR-T cells, with minimal residual disease (MRD) in bone marrow being zero. Two patients have remained as CR for over 60 days and 1 patient has remained as CR over 45 days. Two patients experience no CRS nor ICANs during the treatment and one patient experience grade 1 CRS and no ICANs. No other adverse events were observed.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. An isolated chimeric antigen receptor (CAR) molecule comprising an antigen binding domain, a hinge and transmembrane domain, a costimulatory domain, and an intracellular signaling domain, wherein the hinge and transmembrane domain comprises the amino acid sequence of SEQ ID NO:2.

2. The isolated CAR of claim 1, wherein the hinge and transmembrane domain is encoded by a nucleotide sequence comprising the sequence of SEQ ID NO:1.

3. The isolated CAR of claim 1, wherein the antigen binding domain is a single chain antibody or single chain antibody fragment.

4. The isolated CAR of claim 1, wherein the antigen binding domain binds to a target antigen selected from the group consisting of CD19, CD20, CD22, CD33, CD123, BCMA, CLL1, CD7, CS1, CEA, AFP, PSMA, GPC3, GD2, EGFRVIII, NKG2D, Mesothelin, Claudin 18.2, ROR3, and Muc1.

5. The isolated CAR of claim 1, wherein the antigen binding domain comprises a scFv that binds to CD19, said scFv comprises the amino acid sequences of SEQ ID NOs:4 and 6.

6. The isolated CAR of claim 1, wherein the costimulatory domain comprises a functional signaling domain of a protein selected from the group consisting of OX40, CD2, CD27, CD28, CDS, ICAM-1, LFA-1 (CD11a/CD18), ICOS (CD278), and 4-1BB (CD137).

7. The isolated CAR of claim 1, wherein the intracellular signaling domain comprises an intracellular signaling domain of CD3 zeta, or FcR gamma, or a functional fragment thereof.

8. A nucleic acid construct comprising one or more nucleic acid sequences, said nucleic acid sequences encode the isolated CAR of claim 1, or fragments thereof.

9. An expression vector comprising the nucleic acid construct of claim 8.

10. A cell comprising the expression vector of claim 9.

11. The cell of claim 10, wherein the cell is an immune cell.

12. The cell of claim 11, wherein the immune cell is a T cell.

13. A composition comprising the cell of claim 10 and a pharmaceutically acceptable carrier.


SEQUENCE LISTING (12 sequences)

SEQ ID NO: 1 - DNA, 252 bp, synthetic construct
  source 1..252: mol_type = other DNA; note = modified CD8 hinge and transmembrane domain
gcggccgcattcgtgccggtcttcctgccagcgaagcccaccacgacgccagcgccgcgaccaccaacaccggcgcccaccatcgcgtcgcagcccctgtccctgcgcccagaggcgtgccggccagcggcggcagtgcacacgagggggctggacttcgcctgtgatatctacatctgggcgcccttggccgggacttgtggggtccttctcctgtcactggttatcaccctttactgcaaccacaggaac

SEQ ID NO: 2 - protein, 84 aa, synthetic construct
  source 1..84: note = modified CD8 hinge and transmembrane domain
AAAFVPVFLPAKPTTTPAPRPPTPAPTIASQPLSLRPEACRPAAAVHTRGLDFACDIYIWAPLAGTCGVLLLSLVITLYCNHRN

SEQ ID NO: 3 - DNA, 321 bp, synthetic construct
  source 1..321: mol_type = other DNA; note = anti-CD19 scFv VH
gatattcaaatgacccagtccccgtcctccctgagtgcctccgtcggtgaccgtgttacgattacctgccgtgcgagccaagacatctctaaatacctgaactggtatcagcaaaaaccggatcaggcaccgaaactgctgatcaaacatacctcacgtctgcactcgggtgtgccgagccgctttagtggttccggctcaggtaccgattacaccctgacgatcagctctctgcagccggaagactttgccacgtattactgccagcaaggtaataccctgccgtatacgttcggccaaggtaccaaactggaaatcaaa

SEQ ID NO: 4 - protein, 107 aa, synthetic construct
  source 1..107: note = anti-CD19 scFv VH
DIQMTQSPSSLSASVGDRVTITCRASQDISKYLNWYQQKPDQAPKLLIKHTSRLHSGVPSRFSGSGSGTDYTLTISSLQPEDFATYYCQQGNTLPYTFGQGTKLEIK

SEQ ID NO: 5 - DNA, 360 bp, synthetic construct
  source 1..360: mol_type = other DNA; note = anti-CD19 scFv VL
gaagtgcagctggttgaaagcggtggtggtctggttcaaccgggtcgttccctgcgtctgtcatgtacggcgagtggtgtctccctgccggactatggcgtgtcctggattcgtcagccgccgggtaaaggcctggaatggattggtgtcatctggggcagtgaaaccacgtattacaactcggccctgaaaagccgtttcaccatctctcgcgataacagtaaaaatacgctgtacctgcagatgaatagcctgcgcgcggaagacaccgccgtttactactgcgcaaaacattactactacggtggcagctatgctatggattactggggtcaaggcacgctggtcaccgtttcgtca

SEQ ID NO: 6 - protein, 120 aa, synthetic construct
  source 1..120: note = anti-CD19 scFv VL
EVQLVESGGGLVQPGRSLRLSCTASGVSLPDYGVSWIRQPPGKGLEWIGVIWGSETTYYNSALKSRFTISRDNSKNTLYLQMNSLRAEDTAVYYCAKHYYYGGSYAMDYWGQGTLVTVSS

SEQ ID NO: 7 - DNA, 45 bp, synthetic construct
  source 1..45: mol_type = other DNA; note = scFv linker
ggtggcggtggctcgggcggtggtgggtcgggtggcggcggatct

SEQ ID NO: 8 - protein, 15 aa, synthetic construct
  source 1..15: note = scFv linker
GGGGSGGGGSGGGGS

SEQ ID NO: 9 - DNA, 63 bp, synthetic construct
  source 1..63: mol_type = other DNA; note = signal peptide
atggccttaccagtgaccgccttgctcctgccgctggccttgctgctccacgccgccaggccg

SEQ ID NO: 10 - protein, 21 aa, synthetic construct
  source 1..21: note = signal peptide
MALPVTALLLPLALLLHAARP

SEQ ID NO: 11 - DNA, 462 bp, synthetic construct
  source 1..462: mol_type = other DNA; note = 4-1BB, CD3-zeta signaling domains
aaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 12 - protein, 154 aa, synthetic construct
  source 1..154: note = 4-1BB, CD3-zeta signaling domains
KRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR